(19) United States
(12) Patent Application Publication
(10) Pub. No.: US 2011/0002852 A1
(43) Pub. Date: Jan. 6, 2011

(54) STEM CELLS FOR USE IN LOCATING AND TARGETING TUMOR CELLS

(76) Inventors: Michael Chopp, Southfield, MI (US); Zhenggang Zhang, Troy, MI (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 12/882,718

(22) Filed: Sep. 15, 2010

Related U.S. Application Data
(63) Continuation of application No. 11/324,978, filed on Jan. 3, 2006.
Application No. 11/324,978 is a continuation-in-part of application No. PCT/US04/21365, filed on Jul. 2, 2004.
(60) Provisional application No. 60/485,164, filed on Jul. 3, 2003.

(51) Int. Cl. A61K 49/00; A61K 35/12; A61P 35/00

(52) U.S. Cl. 424/9.1; 424/93.7

(57) ABSTRACT

A composition for locating tumors, the composition includes stem cells. Stem cells for use in locating and treating tumors. A method of locating and treating a tumor by administering to a patient an effective amount of stem cells, wherein the stem cells locate and subsequently treat a tumor.

### CROSS-REFERENCE TO RELATED APPLICATIONS

This application is a Continuation-in-Part of International Patent Application PCT/US04/21365, filed Jul. 2, 2004, which claims the benefit of priority to U.S. Provisional Patent Application No. 60/485,164, filed Jul. 3, 2003, both of which are incorporated herein by reference.

### BACKGROUND OF THE INVENTION

1. Field of the Invention

Generally, the present invention provides a method for locating and treating tumor cells. More specifically, the present invention provides a method for using cells including, but not limited to bone marrow stromal cells to locate and treat tumor cells.

2.Description of the Related Art

In the past, the cure rate for malignant brain tumors has been virtually zero. This is partly due to the size to which the tumor must grow before its presence is diagnosed. If tumors can be detected while still very small in size, or as small clusters of tumor cells, they can be precisely located and removed by the surgical procedures described hereinafter or destroyed by delivery of tumor cytotoxic agents. The amount of cancer material that might be left after such therapies is so small that precisely administered adjuvant therapy, local irradiation, chemotherapy, immuno therapy, etc., may be satisfactory additional treatments. However, there are no non-invasive methods known to locate and identify small clusters of tumor cells.

Surgical procedures are not always applicable for treatment of tumor. It would therefore be beneficial to develop a method of locating a tumor and subsequently treating the tumor without surgical techniques. In other words, it would be beneficial to develop effective methods for the localization and treatment of cancer that does not require surgery. Surgical procedures are employed to remove bulk tumors visible to the eye. However, small clusters of cells, often present after the removal of the bulk tumor, are not amenable to surgical resection.

Despite advances in therapy, morbidity and mortality of malignant brain tumors remain high (Dunn and Black, 2003; Noble, 2000). The highly invasive nature of these tumor cells in normal neural tissue makes them difficult to eradicate (Dunn and Black, 2003; Noble, 2000). Using neural stem cells as therapeutic delivery vehicles, several studies reported that neural stem cells can target tumor mass and invasive satellite tumor cells and promote tumor regression (Aboody et al., 2000; Benedetti et al., 2000; Ehtesham et al., 2002; Lee et al., 2003). The results generated considerable excitement for treatment of malignant brain tumor (Dunn and Black, 2003; Noble, 2000). To date, the use of neural stem cells for targeting and treatment of brain tumor has been restricted to embryonic and neonatal cell populations (Aboody et al., 2000; Benedetti et al., 2000; Ehtesham et al., 2002; Lee et al., 2003). There are no studies in which adult neural stem cells have been employed to target brain tumor. There was previously demonstrated that neural progenitor cells derived from the adult subventricular zone (SVZ) migrate towards infarct boundary regions when grafted into stroke brain in the rat (Zhang et al., 2003b).

Current understanding of neural stem cells targeting brain tumor cells has been derived mainly from regional measurements of labeled embryonic grafted cells using histological and immunohistological methods (Aboody et al., 2000; Benedetti et al., 2000; Ehtesham et al., 2002; Lee et al., 2003). Magnetic resonance imaging (MRI) offers a noninvasive dynamic method for evaluating magnetically-labeled cells in the host brain (Bulte et al., 2002; Zhang et al., 2003a,b).

Additionally, several groups recently demonstrated that embryonic neural stem cells are attractive candidates for treatment of malignant gliomas in mice and rats (Aboody et al., 2000; Benedetti et al., 2000; Ehtesham et al., 2002; Lee et al., 2003). When genetically modified neural stem cells are injected intraparenchymally, intraventricularly, or intravenously, these cells are able to migrate towards tumor mass, promote tumor regression, and prolong survival in animals with implantation of various glioma cell lines (Aboody et al., 2000; Benedetti et al., 2000; Ehtesham et al., 2002; Lee et al., 2003). However, these data have been derived mainly from regional measurements of labeled grafted cells using histological and immunohistological methods (Aboody et al., 2000; Benedetti et al., 2000; Ehtesham et al., 2002; Lee et al., 2003). To further assess interaction between grafted neural stem cells and established tumor in the host brain, a method for noninvasive and dynamic tracking-grafted neural stem cells is required.

It would therefore be beneficial to develop a method and composition for non-invasively locating and treating tumor cells.

### SUMMARY OF THE INVENTION

According to the present invention, there is provided a composition for locating tumors, the composition including stem cells. Stem cells for use in locating and treating tumors are also provided. There is provided a method of locating and treating a tumor by administering to a patient an effective amount of stem cells, wherein the stem cells locate and subsequently treat a tumor.

### DESCRIPTION OF THE DRAWINGS

Other advantages of the present invention are readily appreciated as the same becomes better understood by reference to the following detailed description, when considered in connection with the accompanying drawings wherein:

FIGS. 1A and B are photographs showing that genetically modified MSCs are effective in treating brain tumors;

FIG. 2 shows in vivo activation of NK cell activity in response to the IL-12 secreted by transfected 32DIL-12 cells was measured in a cell cytotoxicity assay using Cr⁵¹-labeled NK-sensitive YAC-1 cells; and

FIG. 3 NK assay was repeated substituting U87 and 4T1 cells for YAC-1 cells.

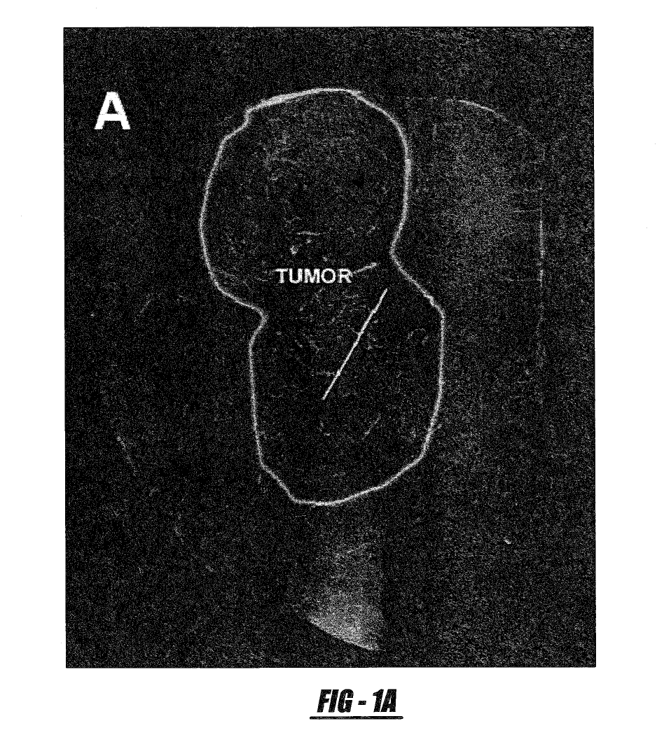

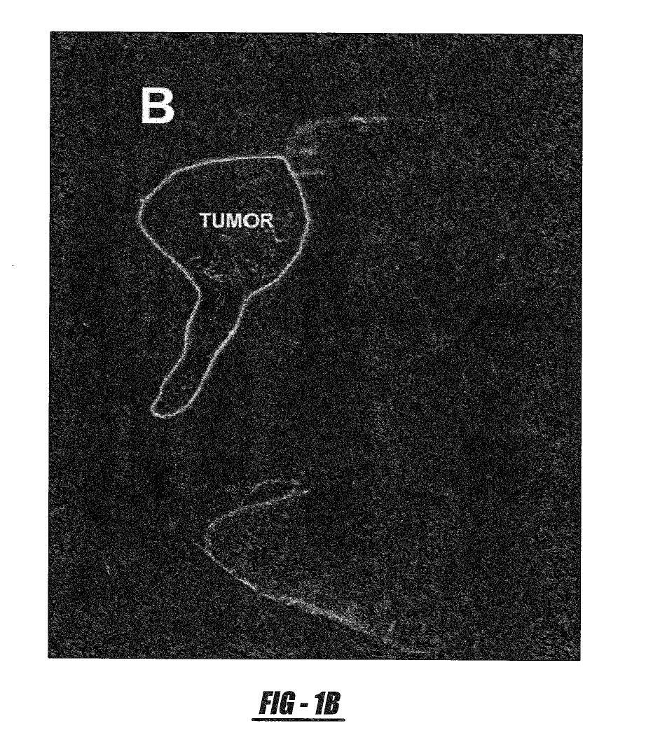

### DETAILED DESCRIPTION OF THE PREFERRED EMBODIMENTS

Generally, the present invention provides a method and composition for locating, and subsequently treating, tumors. More specifically, the present invention provides a method and composition of locating, and subsequently treating tumors, wherein the composition includes scout cells.

The phrase “tumor cells”, as used herein, is intended to include, but is not limited to, at least one cancerous cell or growth.

The phrase “scout cells” as used herein includes, but is not limited to, MSC cells. The MSC cells can be engineered in any manner known to those of skill in the art. Thus, through various genetic engineering methods including, but not limited to, transfection, deletion, and the like, MSC cells can be engineered in order to increase their likelihood of survival or for any other desired purpose.

A stem cell is a generalized mother cell whose descendants specialize into various cell types. Stem cells have various origins including, but not limited to, embryo, bone marrow, liver, stromal, fat tissue, and other stem cell origins known to those of skill in the art. These stem cells can be placed into desired areas as they naturally occur, or can be engineered in any manner known to those of skill in the art. Thus, through various genetic engineering methods including, but not limited to, transfection, deletion, and the like, stem cells can be engineered in order to increase their likelihood of survival or for any other desired purpose.

Stem cells are capable of self-regeneration when provided to a human subject in vivo, and can become lineage-restricted progenitors, which further differentiate and expand into specific lineages. As used herein, “stem cells” refers to human marrow stromal cells and not stem cells of other cell types. Preferably, “stem cells” refers to human marrow stromal cells.

The term “stem cell” or “pluripotent” stem cell are used interchangeably to mean a stem cell having (1) the ability to give rise to progeny in all defined hematopoietic lineages, and (2) stem cells capable of fully reconstituting a seriously immunocompromised host in all blood cell types and their progeny, including the pluripotent hematopoietic stem cell, by self-renewal.

Bone marrow is the soft tissue occupying the medullary cavities of long bones, some haversian canals, and spaces between trabeculae of cancellous or spongy bone. Bone marrow is of two types: red, which is found in all bones in early life and in restricted locations in adulthood (i.e. in the spongy bone) and is concerned with the production of blood cells (i.e. hematopoiesis) and hemoglobin (thus, the red color); and yellow, which consists largely of fat cells (thus, the yellow color) and connective tissue.

As a whole, bone marrow is a complex tissue including hematopoietic stem cells, red and white blood cells and their precursors, mesenchymal stem cells, stromal cells and their precursors, and a group of cells including fibroblasts, reticulocytes, adipocytes, and endothelial cells which form a connective tissue network called “stroma”. Cells from the stroma morphologically regulate the differentiation of hematopoietic cells through direct interaction via cell surface proteins and the secretion of growth factors and are involved in the foundation and support of the bone structure.

Studies using animal models have suggested that bone marrow contains “pre-stromal” cells that have the capacity to differentiate into cartilage, bone, and other connective tissue cells. (Beresford, J. N.: Osteogenic Stem Cells and the Stromal System of Bone and Marrow, Clin. Orthop., 240:270, 1989). Recent evidence indicates that these cells, called pluripotent stromal stem cells or mesenchymal stem cells, have the ability to generate into several different types of cell lines (i.e. osteocytes, chondrocytes, adipocytes, etc.) upon activation. However, the mesenchymal stem cells are present in the tissue in very minute amounts with a wide variety of other cells (i.e. erythrocytes, platelets, neutrophils, lymphocytes, monocytes, eosinophils, basophils, adipocytes, etc.), and, in an inverse relationship with age, they are capable of differentiating into an assortment of connective tissues depending upon the influence of a number of bioactive factors.

The present invention provides a method of locating tumor cells. The method functions by administering scout cells to an individual who may have cancer cells and then monitoring the activity/presence of the scout cells. The scout cells can be monitored in any manner known to those of skill in the art. For example, the scout cell can include labels that can be monitored via MR, CT, SPECT, GAMMA CAMERA, and other optical imaging devices. Specifically, the scout cells can include, as an example and not as a limitation, ferromagnetic particles that can be inserted into the cells without altering the activity of the cells. The scout cells containing the ferromagnetic particles can then be non-invasively monitored as the scout cells travel throughout an individual's body. The cells then locate tumor cells. Preferably, the scout cells are designed to further alter the tumor cells or alter the environment surrounding the tumor cells so as to cause apoptosis or necrosis of the tumor cell(s).

The scout cells of the present invention can be used in two general manners. First, the scout cells can be used to locate tumor cells as disclosed above, which cells can then be treated using known methods. Such methods include, but are not limited to, radiation therapy and localized chemotherapy. Second, the scout cells can be used to locate tumor cells and subsequently treat the tumor cells. In other words, the scout cells can genetically engineered to both seek out and destroy the tumor cells that are located and be genetically and or virally engineered to destroy the tumor cells that are located. An example of such an alteration includes, but is not limited to, transfecting the cells with toxic genes, such as bax and IL-12, or inserting into the cells a virus that cause tumor cell death.

One embodiment of the present invention utilizes IL12-MSC therapy that can dramatically inhibit tumor growth in animals previously implanted with glioma cells. For example, IL12, an immunomodulatory cytokine, is known to be able to thwart tumor growth; however, systemic IL12 has a high toxicity and poor localization to a tumor region. Marrow Stromal Cells are present in an abundant supply, no immunosuppression is required, have highly specific migratory capability, and have selective localization to areas of brain pathology (peritumoral area). Combining the power of two highly potent agents by linking the anti-tumor effects of IL12 with the homing ability of MSC delivery system provides an extremely effective glioma therapy.

In order to cause cell death, expression vectors can be used to introduce the coding sequence of the cell death inducing genes into a cell. Such vectors generally have convenient restriction sites located near the promoter sequence to provide for the insertion of nucleic acid sequences. Transcription cassettes can be prepared comprising a transcription initiation region, the target gene or fragment thereof, and a transcriptional termination region. The transcription cassettes can be introduced into a variety of vectors, e.g. plasmid; retrovirus, lentivirus; adenovirus; and the like, where the vectors are able to transiently or stably be maintained in the cells, usually for a period of at least about one day, and preferably for a period of at least several days to several weeks. Scout cells can also be infected with a virus to destroy the tumor. Methods that localize the agent to the particular targeted tissues are of interest.

DNA constructs can also be used for altering the scout cells. The DNA constructs preferably include at least a portion of the cell death-inducing gene with the desired genetic modification, and include regions of homology to the target locus. Conveniently, markers for positive and negative selection are included. Methods for generating cells having targeted gene modifications through homologous recombination are known in the art. For various techniques for transfecting mammalian cells, see Keown et al. (1990) Methods in Enzymology 185:527-537.

Scout cells can be administered subcutaneously, parenterally including intravenous, intraarterial, intramuscular, intraperitoneally, and intranasal administration as well as with intrathecal and infusion techniques.

The dosage of the scout cells varies within wide limits and is fitted to the individual requirements in each particular case as can be determined by one of skill in the art. In general, in the case of parenteral administration, it is customary to administer from about 0.01 to about 5 million cells per kilogram of recipient body weight. The number of scout cells used depends on the weight and condition of the recipient, the number of or frequency of administrations, and other variables known to those of skill in the art. The scout cells can be administered by a route that is suitable for the suspected location of the tumor to be located and treated. The scout cells can be administered systemically, i.e., parenterally, by intravenous injection, intraarterial injection, or can be targeted to a particular tissue or organ, such as bone marrow. The scout cells can be administered via a subcutaneous implantation of cells or by injection of scout cells into connective tissue, for example muscle. Further, devices currently exist that allow delivery of scout cells. Examples of such devices include, but are not limited to gene guns and other similar devices.

The cells can be suspended in an appropriate diluent, at a concentration of from about 0.01 to about 5×10⁶ cells/ml. Suitable excipients for injection solutions are those that are biologically and physiologically compatible with the cells and with the recipient, such as buffered saline solution or other suitable excipients. The composition for administration must be formulated, produced, and stored according to standard methods complying with proper sterility and stability.

Unless otherwise stated, genetic manipulations are performed as described in Sambrook and Maniatis, MOLECULAR CLONING: A LABORATORY MANUAL, 2nd Ed.; Cold Spring Harbor Laboratory Press, Cold Spring Harbor, N.Y. (1989).

The present invention is advantageous over all currently existing treatments because there are no known side effects and the treatment is relatively non-invasive. The advantages offered by the present invention is the ability to find tumor cells and treat the tumor cells found in a relatively non-invasive manner.

The present invention can replace many current surgical therapies and pharmacological therapies. The present therapy can treat tumors that are not treatable by any of the therapies disclosed in the prior art. Additionally, the present invention is applicable in both the human medical environment and veterinary setting.

The method and composition of the present invention are exemplified in the Examples included herein. While specific embodiments are disclosed herein, they are not exhaustive and can include other suitable designs that vary in design and methodologies known to those of skill in the art. Basically, any differing design, methods, structures, and materials known to those skilled in the art can be utilized without departing from the spirit of the present invention.

### EXAMPLES

### Methods

General methods in molecular biology: Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et at (eds)

Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

General methods in immunology: Standard methods in immunology known in the art and not specifically described are generally followed as in Stites et al.(eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

### Delivery of Therapeutics

The cells of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the cells of the present invention can be administered in various ways. It should be noted that it can be administered as the cells or as pharmaceutically acceptable salt and can be administered alone or as an active ingredient in combination with pharmaceutically acceptable carriers, diluents, adjuvants and vehicles. The cells can be administered orally, subcutaneously or parenterally including intravenous, intraarterial, intramuscular, intraperitoneally, and intranasal administration as well as intrathecal and infusion techniques. Implants of the cells are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness. The doses can be single doses or multiple doses over a period of several days, but single doses are preferred.

The doses can be single doses or multiple doses over a period of several days. The treatment generally has a length proportional to the length of the disease process and drug effectiveness and the patient species being treated.

When administering the cells of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for cells compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells.

Sterile injectable solutions can be prepared by incorporating the cells utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the cells utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196.Many other such implants, delivery systems, and modules are well known to those skilled in the art.

### EXAMPLE 1

Experiments were performed in which IL-12 transfected marrow stromal cells (MSC) were intravenously administered to Nude rats 7 days after U87 glioma cell implantation into the brain. Rats treated with IL-12 MSC (n=7) exhibited small tumor (FIG. 11B) as compared with control rats (n=7) without IL-12 MSC (FIG. 11A). These data demonstrate that genetically modified MSC is effective for treatment of brain tumor.

### EXAMPLE 2

### IL12-Transfected Marrow Stromal Cells: Therapies For Malignant Glioma

### Experiment Design

22 Fisher rats were each implanted with 9 L gliosarcoma cells (50,000 cells each) using standard stereotactic landmarks. The rats were divided into three experimental groups as follows: Group I: Tumor implantation only, no therapy (controls); Group II: Tumor+intra-carotid injection of MSCs alone; and Group III: Tumor+intra-carotid injection of IL12-transfected MSCs.

Analysis of cell localization/tracking, MR imaging comparison studies, histopathology/volumetric analysis, and VEGF/angiogenesis analysis were also performed.

### Tumor Implantation

All Fisher rats underwent standard sterile technique and xylaxine/ketamine anesthesia, followed by small right frontal incision and craniotomy. Specifically-designed Kopf stereotactic head frame and Hamilton syringe containing 50,000 tumor cells were each slowly injected into the right frontal cortex: 3.0 mm right of midline, 2.5 mm anterior to bregma, 2.5 mm deep.

### Intracarotid Injection

Both experimental groups (II and III) underwent standard surgical anesthesia (xylazine/ketamine) and sterile technique to expose the carotid artery seven days after tumor implantation. The carotid artery was exposed and cannulated. Group II was administered a single IA injection of MSCs alone (2×10⁶ cells). Group III was administered a single IA injection of IL12-transfected MSCs (2×10⁶ cells).

At seven days post-treatment, all animals again underwent dynamic MRI for cell tracking and tumor measurement.

### Results

Fisher rats were implanted with 9L gliosarcoma cells. IL-12 MSCs at a dose of 2×10⁶ were administered arterially via the carotid artery at seven days after tumor implantation. Dynamic MRI methods were employed to measure the tumor volume at 7, 10 and 14 days after tumor implantation, respectively. Animals were sacrificed at three weeks after tumor implantation. The MRI and histological data indicated that the IL-12 MSCs significantly inhibit the tumor growth and decrease average tumor volume by approximately 75% (p<0.001), with 30% of the treated animals exhibiting no MRI-detectable tumor mass whatsoever.

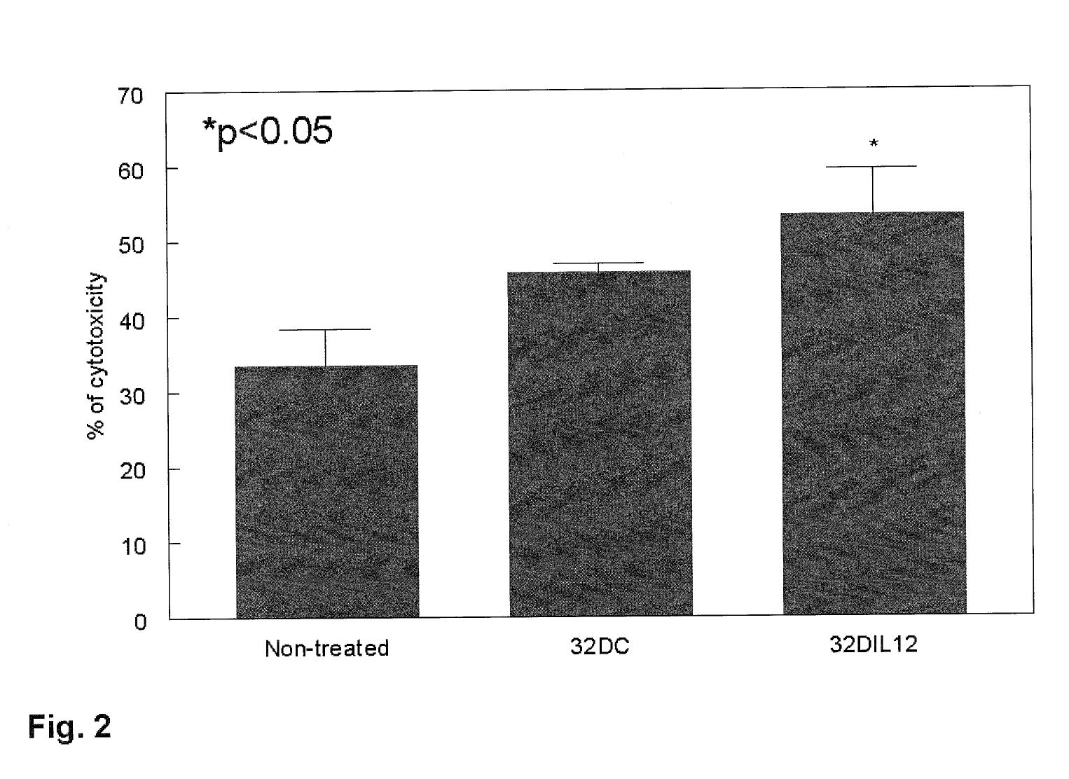

The in vivo activation of NK cell activity in response to the IL-12 secreted by transfected 32DIL-12 cells was measured in a cell cytotoxicity assay using Cr⁵¹-labeled NK-sensitive YAC-1 cells. 2×10⁶ 32DIL-12 cells were administered (i.v.) into the nude rats, and spleen samples were removed at 24 hours after the cell injection. Transfected cells were tested for NK cell-mediated cytotoxicity at effect to target (E:T) ratio of 100:1. The data from a representative experiment (n=4) are shown in FIG. 2. Spleen cell-mediated cytotoxic response against YAC-1 cells of the animals treated by 32DIL-12 cells is significantly higher than in the animals treated with PBS vehicle-control animals (p<0.05).

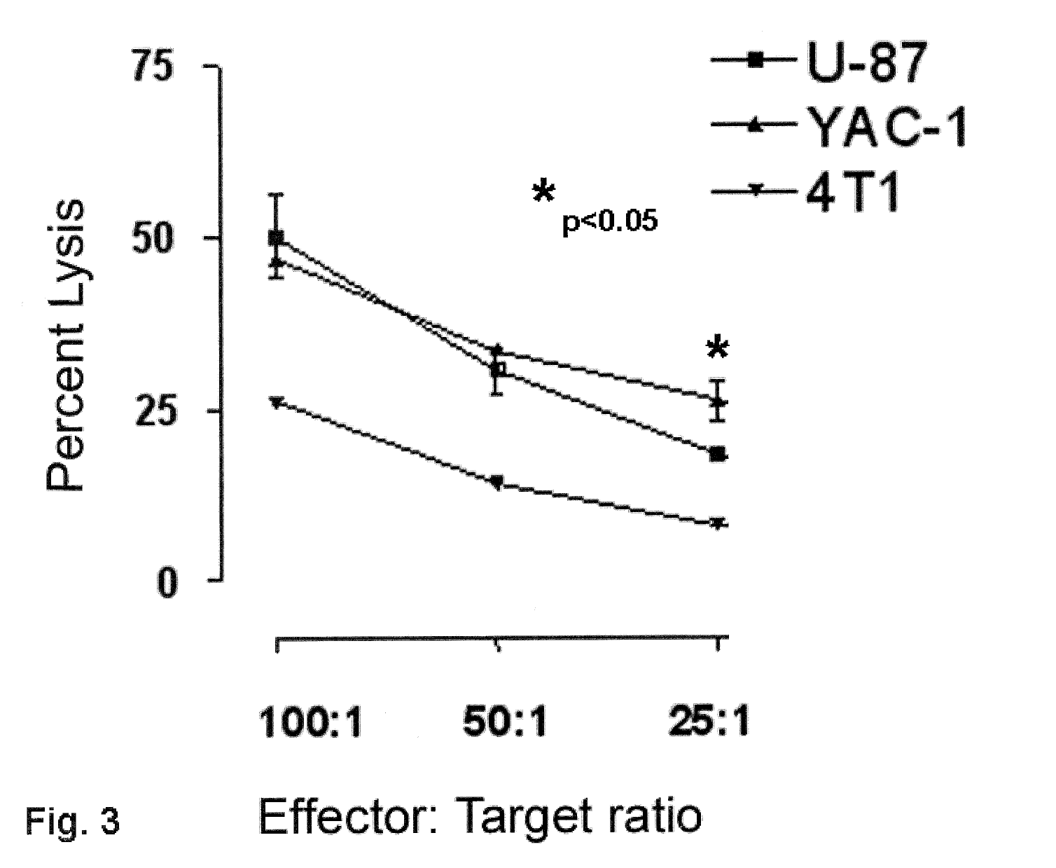

To determine whether U87 tumor cells are NK sensitive, the NK assay was repeated substituting U87 and 4T1 cells for YAC-1 cells. FIG. 3 demonstrates that U87 cell lines exhibit a cytotoxic response that increases with splenic cell concentration. U87 responded similarly to YAC-1. To evaluate U87 tumor response to the cell treatment, the U87 glioma in nude rat model were treated with 32DIL-12 cells, and 32Dc as well as PBS as control groups, respectively. The anti-tumor activity of these cells was measured by using the tumor volume evaluation method. Preliminary data indicates that 32DIL-12 cells significantly inhibit the U87 tumor growth (p<0.001) compared to the nontreated control animals.

To assess the breast tumor response to the cell treatment, the 4T1 breast tumor in nude rat model was treated with two doses of 32DIL-12 cells and PBS as control groups, respectively. The anti-tumor activity of these cells was measured by using the tumor volume evaluation method. The preliminary data indicate that one dose of 32DIL-12 cells (2×10⁶) did not inhibit the tumor growth (p>0.05) compared to the non-treated control animals. However, two doses of 32DIL-12 cells (2×10⁶ each) significantly inhibit the tumor growth (p<0.001) compared to the nontreated control animals.

### Activation of Immune Response Following MSC-IL-12 Therapy of GBM Bearing Mice

Highly infiltrative glioma cells AST11.9-2 and C57/bl6 mouse are employed in this experiment to determine effect of MSC/IL-12 therapy on tumor growth. Additionally, the experiment is designed to analyze whether MSC or MSC/IL-12 therapy diminishes tumor growth following treatment. In order to analyze this, animals were implanted with tumors on day 0, then on day 7, animals received treatment of either MSC, MSC/IL-12 or Mock. Animals were euthanized on either day 10, 13, or 16 following tumor implantation (days 3, 6, and 9 following treatment) and brains were harvested for determination of tumor volume, as well as to characterize the immune components present within the tumor milieu following therapy with MSC vs therapy with MSC/IL-12.

To determine the effect of MSC/IL-12 therapy on development of an anti-tumor immune response, animals were implanted with tumors on day 0, then on day 7, animals received treatment of either MSC, MSC/IL-12 or mock. Animals were euthanized on either day 10, 13 or 16 following tumor implantation (days 3, 6 and 9 following treatment) and blood and spleens harvested for determination of tumor specific immune response and activation of immune components.

To determine whether MSC or MSC/IL-12 therapy results in significant prolongation in survival of GBM tumor bearing mice following treatment, animals were implanted with tumors on day 0, then on day 7, animals received treatment of either MSC, MSC/IL-12 or mock. Animals were followed for survival out to day 180. Animals generally succumbed to tumor development around day 25-30, and thus any animals surviving out 180 were considered cured. However, brains were harvested to determine residual tumor presence.

### Treatment Experiment of Human Glioma U87 With IL-12 Transfected Human MSCs

The experiment evaluated the tumor response to the IL12 treatment that was delivered by human MSCs in nude rat model. By using dynamic MRI and histology methods, the distribution of IL12 and MSCs in tumor, BAT and normal brain tissues were analyzed dynamically. Also, the tumor response to this treatment was studied by dynamically evaluating the tumor size and angiogenesis.

Nude rats are employed in this experiment. Eight animals were implanted with U87 tumor cells treated by MSCs. MRI images (including ex vivo MRI) testing the cell distribution and angiogenesis of these animals were achieved.

Throughout this application, author and year, and patents, by number, reference various publications, including United States patents. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention may be practiced otherwise than as specifically described.

1. A composition for locating and/or treating tumors, said composition comprising stem cells.

2-14. (canceled)

15. A method of locating a tumor by administering to a patient an effective amount of stem cells, wherein the stem cells locate at a site of a tumor.

16-17. (canceled)

18. A method of treating a tumor by administering to a patient an effective amount of stem cells, wherein the stem cells locate and subsequently treat a tumor.

19-20. (canceled)
